(19) United States
(12) Patent Application Publication
(10) Pub. No.: US 2010/0228070 A1
(43) Pub. Date: Sep. 9, 2010

(54) Method for Achieving High Purity Separation and Refinement of Controlling Morphology and Particle Size of 2,6-Dimethylnaphthalene Dicarboxylic Crystals

(75) Inventors: Yang-Ho Cheon, Gyeonggi-do (KR); Young-Gyo Choi, Gyeonggi-do (KR)

Correspondence Address: HARNESS, DICKEY & PIERCE, P.L.C., P.O. BOX 828, BLOOMFIELD HILLS, MI, 48303, US

(73) Assignee: HYOSUNG CORPORATION, Seoul (KR)

(21) Appl. No.: 12/675,408

(22) PCT Filed: Sep. 25, 2008
(86) PCT No.: PCT/KR2008/005684

§ 371 Date: Feb. 26, 2010

(30) Foreign Application Priority Data
Oct. 23, 2007 (KR) 10-2007-0106622

(51) Int. Cl. C07C 7/14

(52) U.S. Cl. 585/817

(57) ABSTRACT

The present invention relates to a method for achieving high purity separation and refinement by controlling morphology and particle size of 2,6-dimethylnaphthalene. And more particularly, the present invention relates to a method for obtaining high purity 2,6-dimethylnaphthalene crystals, in which crystallization is carried out with a solvent that enables the crystals to form a square-platy shape. During the process, crystallization variables such as, stirring speed, cooling speed, solvent and composition ratio, are adjusted to control morphology and particle size of 2,6-dimethylnaphthalene and to remove aggregation, thereby obtaining 2,6-dimethylnaphthalene crystals of high purity.

### TECHNICAL FIELD

The present invention relates to a method for separation and refinement to obtain 2,6-dimethylnaphthalene of high purity by controlling morphology and particle size of 2,6-dimethylnaphthalene crystals.

### BACKGROUND ART

Polyethylene naphthalate (hereinafter referred to as “PEN”) resin is known to excel in several properties such as thermal stability, tensile strength, impact strength and gas permeability, etc. when compared with polyethylene terephthalate (PET) resin. Based on these advantages, PEN is chosen as good materials for fibers, functional polymer, heat-resistant bottles, films and liquid crystal polymer. The most effective and widely known method for producing 2,6-naphthalene dicarboxylic acid (hereinafter referred to as “2,6-NDA”), used as a major material to produce PEN resin, is by oxidation of 2,6-dimethylnaphthalene (hereinafter referred to as “2,6-DMN”). Accordingly, a study on a more efficient method for producing 2,6-DMN is required. In the case of preparing 2,6-NDA by oxidation of 2,6-DMN, the product quality is greatly affected by the purity of 2,6-DMN. That is, a small amount of impurities can greatly deteriorate the properties, such as chromaticity of 2,6-NDA prepared. Therefore, 2,6-DMN having a purity of more than 99 wt % is required to produce 2,6-NDA.

The widely known process for preparing 2,6-DMN comprises the steps of: reacting ortho-xylene and butadiene in the presence of a metal catalyst(Na/K) to form ortho-tolylpentene; cyclizing the prepared ortho-tolylpentene in the presence of a zeolite catalyst to prepare dimethyltetralin; dehydrogenating the prepared dimethyltetralin to produce 1,5-dimethylnaphthalene; and isomerizing 1,5-dimethylnaphthalene thus prepared in the presence of a zeolite catalyst to obtain 2,6-DMN. Accordingly, in order to obtain 2,6-DMN of high purity, a process of separating and purifying 2,6-DMN from dimethylnaphthalene mixtures is required, because the purity of 2,6-DMN obtained by isomerization is less than 20 to 50 wt %.

Dimethylnaphthalne isomers have very close boiling point of about 262.0° C., and thus it is difficult to separate dimethylnaphthalene isomers by conventional distillation method. Thus, separation and purification of 2,6-DMN inevitably results in low yield and high cost with a difficulty in achieving high purity. Several conventional methods for separating and refining DMN isomers are at present widely known, including separation by using complex formation, separation by absorption and separation by recrystallization. These methods, which all lead to separation and refinement of 2,6-DMN, are as follows.

In regards to the separation method by recrystallization listed above, 2,6-DMN is separated with a relatively low cost through the process of crystallization-recrystallization using an appropriate solvent. However, dimethylnaphthalenes are generally known to form eutectic mixtures. For example, 2,6-DMN and 2,7-DMN form eutectic mixtures in a molar ratio of 47.5:52.5. And 2,6-DMN and 2,3-DMN form eutectic mixtures in a molar ratio of 41.5:58.5. In theory, the amount of 2,6-dimethylnaphthalene to be formed is determined depending on the composition of substances. Therefore, highly efficient separation can not be achieved by recrystallization. In addition, separation by recrystallization requires complicated processes and a long process time, but its final purity is relatively low, making the method undesirable.

European Patent Publication No. 0 336 564 A1 (in 1989) discloses a method for separating 2,6-DMN, which comprises three steps of pretreatment, distillation and pressure crystallization of naphthalene-based compound. However, the purity of 2,6-DMN thus separated is less than 98%, which is below the purity level required for the production of 2,6-naphthalnee dicarboxylic acid.

Korean Laid-Open Patent Publication No. 10-2001-33746 teaches a method for producing 2,6-DMN of high purity with a high yield from a mixture of DMN isomers through a series of processes including fractionation, crystallization and absorption, without restricting 2,6-DMN. The above method is characterized in that 2,6-DMN is dissolved in p-xylene and o-xylene as final purifying steps through crystallization, to absorb and separate it. Japanese Laid-Open Patent Application Nos. 1997-301900 and 1997-249586 teach a method for producing 2,6-DMN from a mixture of DMN isomers through crystallization in the presence of a solvent. These methods are directed to industrially advantageous means of suspiration and recovery, because the methods allow maintaining of the product purity at least a predetermined level with stability over a long period of time.

U.S. Pat. No. 5,675,022 describes a method for dynamic melt crystallization using a Sulzer Cemtech apparatus, which is a falling film crystallizer, comprising flowing a molten liquid on a cooled surface, in the form of a liquid film, by means of forced convection. However, this method involves dynamic layer crystallization which disadvantageously requires performing crystallization five times or more through multi-stage (five-stage) crystallization, and use of additional apparatus.

Among these methods, the methods employing crystallization are known to be simplest and most suitable for industrial application. However, the methods employing crystallization have problems of requiring relatively high sums of fixed investment and production costs because the process is relatively complicated, results in a low yield, and makes use of expensive solvents. Particularly, in the case of using a separation process through crystallization, the separation process involves simple cooling and crystallization in most case, and is focused on the process of isomerization or absorption using catalyst, rather than crystallization.

### DISCLOSURE

### Technical Problems

The present invention provides a method for separating and refining 2,6-DMN at a low cost in a continuous, efficient and industrially-advantageous manner, by means of controlling particle size and morphology of 2,6-DMN, the method of which has never been involved in any of the prior arts.

As described above, crystallization is commonly used in prior arts. However, these conventional methods have never involved the crystal manufacturing conditions relating to a particle size and morphology, which directly affects purity of crystals. The inventors of the present invention found that a cooling crystallization results in an excessive amount of scale-shaped crystals and aggregation thereof. In that case, refinement of impurities is not achieved to a required level and solvent viscosity is increased, thereby making it difficult to filtrate and to remove mother liquid contained on the surface. For these reasons, the efficiency of separation and refinement of 2,6-DMN obtained by the prior methods greatly decreases.

Thus, the object of the present invention is to provide conditions of producing 2,6-DMN crystals having a particle size of 150 to 300 μm and a polyhedral, square platy-like shape in the crystallization process by using a specific solvent. In addition, the method for refinement according to the present invention enables removing other isomers and impurities contained in 2,6-DMN.

### Technical Solution

In order to solve the above problems, the present invention provides a method for separating and refining 2,6-DMN having a purity of 99.0 wt % from a mixture of dimethylnaphthalene isomers, in which solvents, crystallization temperatures, stirring and cooling speed of the crystallization unit are adjusted to thereby control morphology of 2,6-DMN.

According to the present invention, the solvent is preferably methanol or ethanol.

According to the present invention, the temperature of the crystallization unit is adjusted to −10 to −5° C.

According to the present invention, the stirring speed of the crystallization unit is set at 30 to 100 rpm.

According to the present invention, the cooling speed is set at 0.01 to 1° C./min.

According to the present invention, the growth rate of the crystal in the crystallization unit is adjusted to 1×10⁻⁹ to 1×10⁻⁷ m/s.

According to the present invention, the crystallization unit has an external jacket, and the temperature thereof is preferably adjusted in the range of −30° C. to −5° C.

According to the present invention, the mixture of dimethylnaphthalene isomers and solvents consist of 0.5 to 10 wt % of 2,6-DMN, 87 to 98 wt % of ethanol and less than 60 wt % of other DMN compounds.

### Advantageous Effects

Accordingly, the present invention provides a method for separation and refinement of high purity 2,6-DMN having a purity of more than 99.0 wt % through a one-time crystallization process by controlling morphology and particle size of 2,6-DMN crystals. The separation and refinement process of the present invention is carried out by fusion, the temperature of which is about one-fifth of that of the evaporation heat, thereby saving energy and separating high purity 2,6-DMN at high yield by simply manipulating solid-liquid separation. In addition, the present invention is economically advantageous in that the method for separation and refinement utilizes a simple apparatus and a simplified operation, leading to reduced fixed investment and production costs. And solution crystallization is carried out as an additional process, thereby enabling effective separation of high purity 2,6-DMN.

### BRIEF DESCRIPTION OF DRAWINGS

FIG. 1 is photograph showing crystal morphology of 2,6-dimethylnaphthalene varying depending on different solvents used in the crystallization method of the present invention.

FIG. 2 is a graph of tertiary component showing a region where 2,6-dimethylnaphthalene crystal of a square-platy shape is formed by crystallization according to the mixing ratio of ethanol as a solvent, 2,6-dimethylnaphthalene and other dimethylnaphthalenes.

FIG. 3 is a photograph showing 2,6-dimethylnaphthalene crystal forms by crystallization according to the mixing ratios (N1-N5) of solvents and mixed materials.

FIG. 4 shows morphology of 2,6-dimethylnaphthalene crystals depending on varying stirring speed according to the method of the present invention.

FIG. 5 shows morphology of 2,6-dimethylnaphthalene crystals depending on varying crystallization temperatures according to the present invention.

### BEST MODE

2,6-DMN of the present invention is obtained with the following steps of: appropriately mixing and dissolving a mixture of dimethylnaphthalene isomers and a selected solvent in accordance with a composition ratio; performing crystallization by continuously transferring the resulting mixture to a crystallization unit; and separating 2,6-DMN crystals produced through proper control from a mother liquid. According to the method of the present invention, a one-time crystallization process enables the separation and refinement of high purity 2,6-DMN having a purity of more than 99.0 wt %.

Generally in the crystallization process, crystal morphology, which is one of important features of crystals, should be varied depending on its usage. Further, controlling crystal morphology is a critical means for separating a specific substance from a mixture. Particularly, by crystallization, isomers having similar boiling points can be easily separated, which is usually a difficult process in the case of distillation or other separation methods. For example, in order to facilitate handling or packaging of crystals prepared, a specific ball-shape crystal is required to improve a stream of crystal particles. And at times, crystals of a platy shape or a needle shape are required for a specific purpose. The crystal morphology along with crystal size and crystal size distribution(CSD) is an important factor in determining crystal design and operation of a crystallization unit. Thus, in almost every crystallization process, transforming crystal morphology by a specific method is required to change crystal shapes. Crystal morphology shows only an external shape of a crystal, whereas crystal habits show various shapes formed by a difference in growth rate of each crystal surface. Changing crystal habits is basically a field of interest in crystallization, and the research on which is actively conducted. A new formation or extinction of crystal surfaces can lead to a transformation of external shape of crystals without any change in the internal structure of crystals. And a growth speed of each crystal surface is dependent upon various crystallization conditions. Factors that affect the crystal habit include the types and features of a solvent, the types and contents of impurities, the supersaturation degree and other crystallization conditions. After conducting experiment on crystal habits of 2,6-DMN in a multicomponent mixture, and observing changes in crystal morphology, habits and features, a method for separating and refining 2,6-DMN of high purity has been found.

According to the present invention, the crystallization method using a selected solvent involves forming crystals from saturated solution by adjusting nuclear formation and the growth rate of crystals. By the method, purity and particle size distribution of crystals are adjusted, in which the condition of forming supersaturation from saturated solution serves as an important variable. The purity of crystals is deteriorated by mother liquid or impurities contained in crystals. However, the problem can be solved by adjusting crystallizing features such as nuclear formation, the growth rate of crystals, etc., thereby optimizing the crystallizing conditions. The decreased speed in crystal growth and the increased speed in substance transmission lead to a reduction in impurities contained in the crystals, thereby increasing the purity of crystals. The crystal purity is also affected by crystal morphology. A multifaceted crystal has better purity than a needle-shaped crystal, because a needle-shaped crystal has a large surface area such that it requires a larger amount of mother liquid attached than a multifaceted crystal. And in that case, the crystal is prone to agglomerate, and the impurities can be easily contained in the crystal. The crystal purity is dependent upon the crystal size as well. As the crystal gets smaller, the surface area is increased, thereby making it difficult to remove impure mother liquid attached on the external surface the crystal. In that case, an additional purifying process is required as impurities are prone to remain even after separation of mother liquid from crystals.

According to a crystal growth model, crystals grow by diffusion and surface reaction. A crystal having a desired size, morphology and purity can be obtained by controlling a proper variable relevant to crystal growth, depending on which factor, i.e., diffusion or surface reaction, controls the crystal growth, to thereby increase the crystal purity. The variables which control factors of crystal growth in the crystallization process include cooling and stirring speed, change in the mixing ratio and the amount of solvents to be used, etc. Generally, in the case of using a solvent, the crystal growth is controlled by diffusion rather than surface reaction. In the crystallization process, the important variables that control crystal diffusion are cooling and stirring speed, the type of solvents and the crystallizing time. And by adjusting the variables, crystal morphology of 2,6-DMN is controlled to obtain high purity 2,6-DMN.

According to the method of the present invention, the crystal size is dependent upon a saturation temperature of a solvent in the early stage, stirring speed, the temperature of a refrigerant and the growth rate of crystals, etc. The particle size of a crystal is in inverse proportion to the rate of nuclear formation and in proportion to the rate of the crystal growth. Therefore, adjusting the rate of nuclear formation is highly important in controlling the crystal particle size to thereby control the total number of crystal particles. Accordingly, the present invention discloses a continuous crystallization method for separation and refinement by adjusting growth conditions, particle morphology and particle size of 2,6-DMN crystals to obtain 2,6-DMN of high purity.

Hereinafter, the present invention will be described in detail.

According to the method for producing 2,6-DMN crystals, DMN mixtures and a solvent are fed at a certain influx rate into a material tank to be dissolved. And then the resulting solvent is continuously supplied to a jacket-type crystallization unit equipped with a scrape and arranged in a row. The DMN mixture dissolved in a solvent is crystallized in the crystallization unit by adjusting a refrigerant flowing along a jacket of the crystallization unit. And at the same time, by rotating the scrape, crystals formed are transferred in a suspension state along an outlet of the crystallization unit into a solid-liquid separation device and are refined through the solid-liquid separation device to thereby obtain 2,6-DMN of high purity.

The DMN mixtures above are a mixture containing 10 DMN isomers obtained in the isomerizing process, and compounds of hydrocarbon having high or low boiling points, in which 2,6-DMN, 1,6-DMN, 1,5-DMN and other compounds are contained in respective contents indicated in the following table 1.

TABLE 1
Composition of dimethylnaphthalene isomer
compounds.

| Composition | Content (wt %) |
|---|---|
| 2,6-DMN | 35~45 |
| 1,6-DMN | 44~42 |
| 1,5-DMN | 10~9 |
| Low boiling point compound | 7~3 |
| High boiling point compound | 4~1 |
| Total | 100 |

The mixed materials containing 35˜45 wt % of 2,6-DMN based on the weight of the mixture are dissolved at a crystallization exit temperature of 50-60° C., and fed into the crystallization unit equipped with a scrape. The temperature of crystallization jacket is set at −30° C., and the crystallization unit is circulated with an arrangement in a row to form crystals therein. The crystals formed are then pushed by the rotation of the scrape equipped in the crystallization unit to separate and refine the crystals. In this manner, square-platy 2,6-DMN crystals with a size of 150˜300 μm can be obtained continuously in the cooling crystallization unit equipped with a scrape and arranged in a row.

The inside of the crystallization unit arranged in a row is divided into a quasi-stable area and a growth area, in which the cooling speed is adjusted such that nuclear formation and crystal growth occur simultaneously in a single reactor. The temperature of the external jacket of the crystallization is set at less than −30° C. to form crystals inside the crystallization unit.

The growth rate of the crystals formed is controlled by the cooling speed programmed from the quasi-stable area to the temperature of supersaturation. The crystal growing speed is preferably set at 1×10⁻⁹˜1×10⁻⁷ m/s. 2,6-DMN is discharged after the process of nuclear formation and crystal growth in the crystallization unit with the exit temperature of the crystallization unit set at −10˜−5° C.

When the crystals formed inside are pushed by the rotation of the scrape, the rotating speed of the scrape serves as manipulated variable that determines the crystal size. The rotating speed of the scrape agitator is preferably set at 30-100 rpm.

2,6-DMN of high purity is obtained by continuously producing 2,6-DMN crystals of 150˜30 μm from the crystallization unit, and by removing impure solvents on the surface of the crystals produced using a solid-liquid separation device.

The present invention is advantageously capable of controlling crystal size and morphology by adjusting the growth rate of 2,6-DMN crystals, and further, of refining by easily removing impurities contained in 2,6-DMN. The rate of crystallization can be controlled by adjusting the temperature of a refrigerant, the influx rate and the mixing ratio of solvents and the mixed materials. And square-platy shape particles can be obtained by adjusting the crystallization speed.

Hereinafter the method for separating and refining 2,6-DMN of the present invention will be described in further detail with preferred embodiments.

1. Selecting a Solvent to Control Crystal Morphology

Selecting a solvent is important in that the separation and refinement method according to the present invention involves changing inherent habits of the crystals. A specific solvent enables the crystals to be separated with a constant morphology, thereby allowing high purity. A solvent is directly related to solubility in the crystallization process, and affects not only purity and yield, but a crystal habit as well, thereby allowing crystals to have a certain form. The crystal habit enables separation and refinement of high purity crystals, indicating that selection of a solvent is an important factor in crystallization to have crystals of high purity.

After a solubility test, selected solvents include methanol, ethanol, propanol, butanol, pentanol, hexanol, heptanol, hexane, heptane, acetone, toluene, ortho-xylene, meta-xylene, para-xylene and dimethyltetralin. The method of the present invention employs a certain solvent that enables crystals to grow in a specific form to thereby obtain high purity crystals.

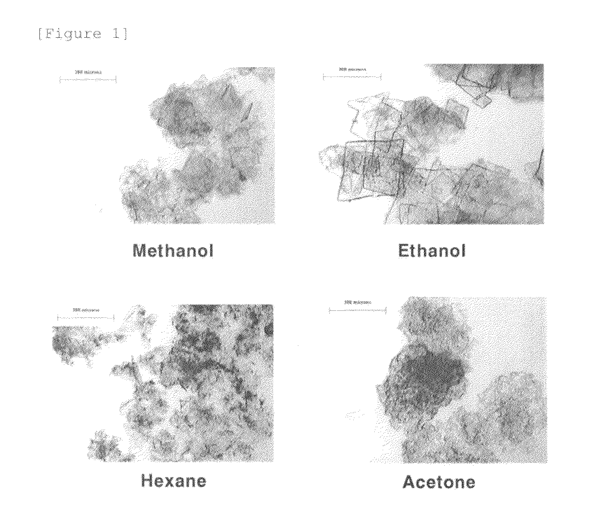

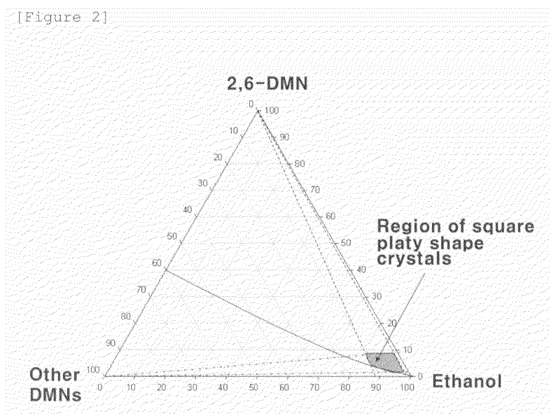

Crystal forms varying depending on different solvents are shown in FIG. 1, and purity changes showing a degree of separation by controlling crystal forms are shown in FIG. 2. As presented in FIG. 1, high purity crystals having a size of 105˜500 μm and a square-platy shape can be obtained by using ethanol and methanol as a solvent.

TABLE 2
Effect of solvents on crystals

| | | | Average purity | |
|---|---|---|---|---|
| | Crystal | Average | of crystals | |
| Solvents used | morphology | crystal size | obtained | Reference |
| Solvents not | A scale shape, | 50~100 μm | 57.3 wt % | The average |
| used | agglomerates | | | value of three |
| | | | | tests |
| Methanol | Square-platy | 50~300 μm | 98.1 wt % | The average |
| | shape | | | value of three |
| | | | | tests |
| Ethanol | Square-platy | 100~500 μm | 99.3 wt % | The average |
| | shape | | | value of three |
| | | | | tests |
| Propanol | Platy shape | 50~300 μm | 95.3 wt % | The average |
| | | | | value of three |
| | | | | tests |
| Butanol | Platy shape | 50~100 μm | 97.1 wt % | The average |
| | | | | value of three |
| | | | | tests |
| Pentanol | Scale shape | 30~70 μm | 90.7 wt % | The average |
| | | | | value of three |
| | | | | tests |
| Hexanol | Scale shap | 10~50 μm | 86.4 wt % | The average |
| | | | | value of three |
| | | | | tests |
| Heptanol | Scale shape | 10~50 μm | 80.0 wt % | The average |
| | | | | value of three |
| | | | | tests |
| Ortho-xylene | Scale shape | 10~30 μm | 75.5 wt % | The average |
| | | | | value of three |
| | | | | tests |
| Para-xylene | Scale shape | 10~30 μm | 63.3 wt % | The average |
| | | | | value of three |
| | | | | tests |
| Meta-xylene | Scale shape | 10~30 μm | 52.2 wt % | The average |
| | | | | value of three |
| | | | | tests |
| Acetone | Scale shape | 30~70 μm | 87.1 wt % | The average |
| | | | | value of three |
| | | | | tests |
| Hexane | Scale shape | 5~100 μm | 89.0 wt % | The average |
| | | | | value of three |
| | | | | tests |
| Heptane | Scale shape | 5~30 μm | 85.0 wt % | The average |
| | | | | value of three |
| | | | | tests |
| Dimethyltetralin | Scale shape | 5~10 μm | 63.2 wt % | The average |
| | | | | value of three |
| | | | | tests |

2. The Mixing Ratio of DMN Mixtures to Control Crystal Morphology of the Present Invention.

The mixing ratio of DMN mixtures is an important variable directly related to morphology, purity and yield of crystals. FIG. 2 is a graph of tertiary component consisting of ethanol as a solvent, 2,6-DMN and other DMN mixtures. As shown in FIG. 2, it was found that only when the percentage of the tertiary component is 0.5 to 10% of 2,6-DMN, 87˜98% of ethanol and other DMN mixtures in the range of 0˜60%, crystals are allowed to have a square-platy shape, thereby obtaining high purity crystals. Crystallization with ethanol as a solvent enables finding a mixing range of solvents that leads to obtaining high purity 2,6-DMN having a purity of more than 99.0 wt % and a square-platy shape.

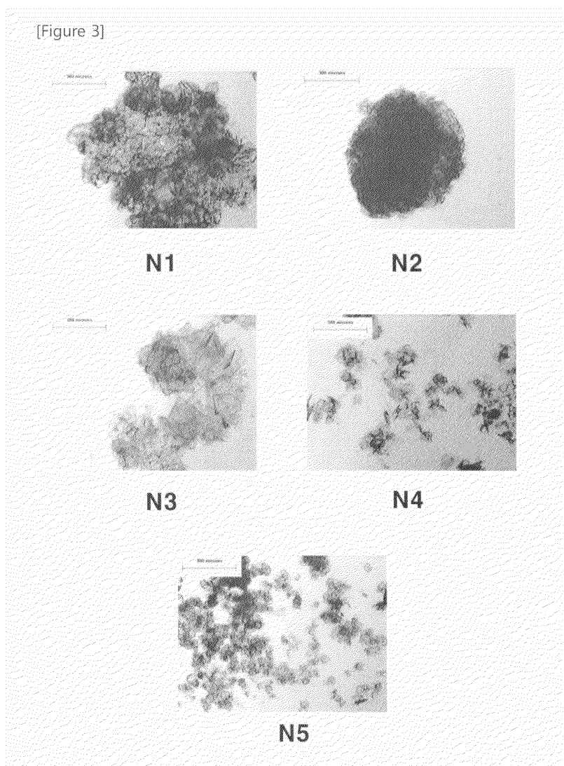

FIG. 3 and Table 3 show an effect of mixing ratio of mixtures under the conditions of a cooling speed of 0.75° C./min, a stirring speed of 100 rpm and a temperature of −10° C. The purity increases in proportion with the increase in the percentage of solvent, but yield of crystals decreases accordingly. The increase in solvent ratio leads to a decrease in the suspension density, thereby preventing the formation of agglomerates, and making the size of crystals smaller. When crystals agglomerate, solvent is contained between crystals with other impurities involved, thereby deteriorating purity. Therefore, in consideration of purity and yield, the crystallization should be performed by using a proper solvent to control crystal morphology, thereby obtaining high purity crystals.

In Table 3, mixing ratio (N) refers to a mixing ratio of ethanol: 2,6-DMN: impurities containing other DMNs. The mixing ratios of mixtures below are each N1=83:7:10, N2=89:4:7, N3=92:3:5, N4=95:2:3 and N5=92:1:2,

TABLE 3
The effect of composition ratio of mixtures

| | mixing ratio | | | | |
|---|---|---|---|---|---|
| | N1 | N2 | N3 | N4 | N5 |
| Purity | 57 wt % | 91 wt % | 99.3 wt % | 99.5 wt % | 99.7 wt % |
| yield | 87% | 94.5% | 93% | 92.5% | 75% |

3. The Effect of Stirring Speed on Controlling Crystal Size of the Present Invention

The purity of crystals deteriorates in the morphology controlling area due to impurities contained depending on the crystal size. Therefore, controlling crystal size is also an important variable.

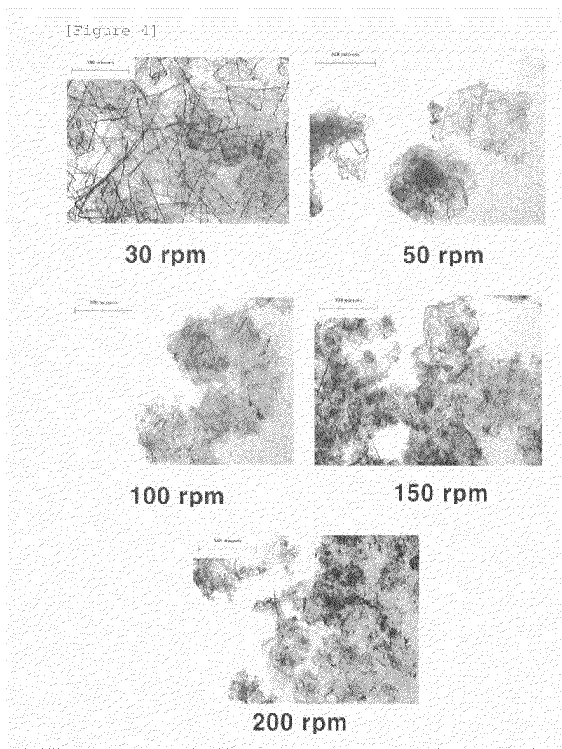

The stirring speed is a critical variable in 2,6-DMN batch cooling crystallization unit. Stirring speed enables homogeneous supersaturation in the entire crystallizatioin unit during the crystallization process. That is, solute molecules are uniformly distributed and temperatures are kept with an ignorable decline during the crystallization process. Furthermore, solid suspension density is maintained in bulk solution to thereby provide a uniform surface area for a uniform growth of crystals. Partially high supersaturation in the crystallization unit may cause a spontaneous formation of nuclides in the early stage, which leads to formation of crystals having a small average size, thereby resulting in enlargement of crystal size distribution, and therefore affecting crystal purity. FIG. 4 and Table 4 show an effect of stirring speed under the condition of 2,6-DMN of 40.3 wt %, crystallization temperature at 0° C., solvent ratio of 20 (a ratio of ethanol to DMN mixtures) and cooling speed=0.75 k/min. According to the result of (a)-(e) of FIG. 4 and Table 4, purity is better as the stirring speed is low. Especially, in the case of no stirring applied, the highest purity is obtained. This is because nuclides are formed at a lower temperature, and the crystals formed in a platy structure are not broken into pieces with no stirring. The stirring speed of more than 100 rpm does not affect the purity and yield, and therefore, does not make a big difference in the crystallization process of 2,6-DMN.

According to the changes in morphology of crystals obtained depending on a varying stirring speed, as the stirring speed gets higher, the size of crystals gets smaller and the crystals are broken into pieces, which results in purity degradation with platy shape crystals broken due to stirring.

Crystallization with a stirring speed of 30˜100 rpm can prevent the crystals from being broken maintain the platy shape of DMN crystals, thereby obtaining high purity DMN.

TABLE 4
The effect of stirring speed

| | Temperature | Purity of | | |
|---|---|---|---|---|
| Stirring | of forming | crystals | | Size of |
| speed | nuclide(° C.) | obtained(wt %) | Yield (%) | crystals (μm) |
| No stirring | 3.0 | 99.7 | 40.0 | 250~500 |
| 30 RPM | 7.0 | 99.3 | 75.0 | 250~300 |
| 50 RPM | 9.0 | 99.1 | 79.0 | 200~300 |
| 75 RPM | 10.0 | 99.1 | 80.0 | 170~250 |
| 100 RPM | 11.0 | 99.0 | 80.0 | 150~250 |
| 150 RPM | 11.0 | 98.7 | 78.0 | 30~150 |
| 200 RPM | 12.0 | 98.8 | 78.0 | 20~150 |
| 300 RPM | 12.0 | 98.6 | 79.0 | 5~100 |

4. The Effect of Cooling Speed on Crystallization of the Present Invention

The most general method for the control of supersaturation in the cooling crystallization unit is by means of cooling speed. Table 5 shows an effect of the cooling speed under the condition of injection composition of 41.2 wt %, solvent ratio of 20 and stirring speed at 100 rpm. As shown in Table 5, as the cooling speed gets higher, the crystal purity deteriorates. This is because a higher cooling speed causes a larger amount of impurities to be contained in the crystals. However, yield is relatively less affected by the cooling speed, because crystallization is hugely affected by the crystallizing temperature.

TABLE 5
the relation of crystallization and cooling speed

| | Crystallizing | | Crystallizing | | Crystallizing | | Crystallizing | |
|---|---|---|---|---|---|---|---|---|
| | temperature | | temperature | | temperature | | temperature | |
| | at 0° C. | | at −5° C. | | at −10° C. | | at −20° C. | |
| Cooling | Purity | Yield | Purity | Yield | Purity | Yield | Purity | Yield |
| speed | (wt %) | (%) | (wt %) | (%) | (wt %) | (%) | (wt %) | (%) |
| 0.1° C./min | 99.7 | 32 | 99.5 | 79 | 99.3 | 81 | 96.5 | 94.0 |
| 0.2° C./min | 99.5 | 31 | 99.3 | 78 | 99.1 | 81 | 95.3 | 93.0 |
| 0.75° C./min | 99.5 | 31 | 99.3 | 75 | 99.0 | 80 | 94.7 | 93.5 |
| 1° C./min | 99.4 | 30 | 99.2 | 73 | 98.9 | 79 | 93.2 | 92.0 |
| 10° C./min | 99.3 | 29 | 98.8 | 71 | 96.8 | 78 | 93.7 | 91.7 |

5. The Effect of Crystallizing Temperature on Crystallization Process

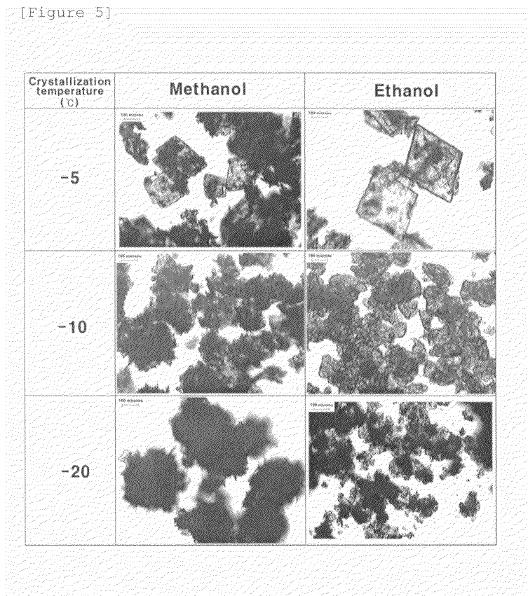

In the cooling crystallization process, the cooling temperature is the most important variable in purity and yield. FIG. 5 and Table 6 show the result on measurement of purity and yield depending on varying temperatures of crystallization under the condition of injection composition of 45 wt %, cooling speed of 0.75° C./min, stirring speed of 1000 rpm and a solvent ratio of 20. As the crystallizing temperature gets lower, the crystals are broken and relatively small-size crystals agglomerate, indicating that agglomeration causes purity degradation.

TABLE 6
the effect of crystallizing temperature

| | Crystallizing temperature (° C.) | | | | | |
|---|---|---|---|---|---|---|
| | 0 | −5 | −10 | −15 | −20 | −30 |
| Purity (wt %) | 99.7 | 99.3 | 99.1 | 98.5 | 97.4 | 96 |
| Yield (%) | 65.0 | 80.0 | 90.0 | 93.0 | 95.0 | 97.0 |

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof. Hereinafter, preferred embodiments will be described.

1. A method for separating and purifying 2,6-dimethylnaphthalene having a purity of more than 99.0 wt % from a mixture of dimethylnaphthalene isomers, wherein solvents, crystallizing temperatures, stirring and cooling speed in the crystallization unit are adjusted to thereby control crystal morphology.

2. The method for separating and purifying 2,6-dimethylnaphthalene according to claim 1, wherein the solvent is methanol or ethanol.

3. The method for separating and purifying 2,6-dimethylnaphthalene according to claim 1, wherein the crystallizing temperature is adjusted to −10 to −5° C.

4. The method for separating and purifying 2,6-dimethylnaphthalene according to claim 1, wherein the stirring speed of the crystallization unit is set at 30 to 100 rpm.

5. The method for separating and purifying 2,6-dimethylnaphthalene according to claim 1, wherein the cooling speed is set at 0.01 to 1° C./min.

6. The method for separating and purifying 2,6-dimethylnaphthalene according to claim 1, wherein the speed of crystal growth rate is adjusted to 1×10⁻⁹ to 1×10⁻⁷ m/s.

7. The method for separating and purifying 2,6-dimethylnaphthalene according to claim 1, wherein the crystallization unit has an external jacket at a temperature adjusted in the range of −30° C. to −5° C.

8. The method for separating and purifying 2,6-dimethylnaphthalene according to claim 1, wherein the mixture of dimethylnaphthalene isomers and solvents consist of 0.5 to 10% of 2,6-DMN, 87 to 98% of ethanol and less than 60% of other DMN compounds.

9. The method for separating and purifying 2,6-dimethylnaphthalene according to claim 1, wherein the crystal has a square-platy shape with a size of 150 to 300 μm.
